(19) United States
(12) Patent Application Publication
(10) Pub. No.: US 2026/0345714 A1
(43) Pub. Date: Nov. 5, 2026

(54) METALLO-BETA-LACTAMASE INHIBITOR AND USE THEREOF

(71) Applicant: NIHON SIZEN HAKKOH CO., LTD., Takayama-shi (JP)

(72) Inventors: Osamu TOKUNO, Aichi (JP); Masahiro NAKANISHI, Gifu (JP)

(73) Assignee: NIHON SIZEN HAKKOH CO., LTD., Takayama-shi (JP)

(21) Appl. No.: 19/139,056

(22) PCT Filed: Dec. 13, 2022
(86) PCT No.: PCT/JP2022/045833

§ 371 Date: Jun. 13, 2025

(51) Int. Cl. A61K 36/736; A61K 31/194; A61K 31/198; A61K 31/401; A61K 31/405; A61K 31/4172; A61K 31/43; A61K 31/685; A61K 31/7016; A61K 31/717; A61K 35/744; A61K 36/03; A61K 36/064; A61K 36/07; A61K 36/16; A61K 36/23; A61K 36/282; A61K 36/31; A61K 36/42; A61K 36/44; A61K 36/48; A61K 36/49; A61K 36/535; A61K 36/605; A61K 36/72; A61K 36/752; A61K 36/78; A61K 36/81; A61K 36/87; A61K 36/888; A61K 36/8962; A61K 36/8967; A61K 36/8994; A61K 36/8998; A61K 36/9066; A61K 36/9068; A61K 38/05; A61K 47/26; A61K 47/36; A61P 31/04

(52) U.S. Cl. CPC A61K 36/736; A61K 31/194; A61K 31/198; A61K 31/401; A61K 31/405; A61K 31/4172; A61K 31/43; A61K 31/685; A61K 31/7016; A61K 31/717; A61K 35/744; A61K 36/03; A61K 36/064; A61K 36/07; A61K 36/16; A61K 36/23; A61K 36/282; A61K 36/31; A61K 36/42; A61K 36/44; A61K 36/48; A61K 36/49; A61K 36/535; A61K 36/605; A61K 36/72; A61K 36/752; A61K 36/78; A61K 36/81; A61K 36/87; A61K 36/888; A61K 36/8962; A61K 36/8967; A61K 36/8994; A61K 36/8998; A61K 36/9066; A61K 36/9068; A61K 38/05; A61K 47/26; A61K 47/36; A61P 31/04; A61K 2236/19

(57) ABSTRACT

A metallo-β-lactamase inhibitor containing, as an active ingredient, a fermented plant product which is a mixture of (a) a koji mold-fermented product of beans and grains including barley, black soybean, red rice, black rice, adzuki bean, adlay, Japanese barnyard millet, foxtail millet, and proso millet, (b) a yeast and/or lactic acid bacterium-fermented product of fruits including mandarin orange, grape, apple, crimson glory vine, peach, persimmon, and the like, (c) a yeast and/or lactic acid bacterium-fermented product of root vegetables and tubers including purple sweet potato, Jerusalem artichoke, carrot, onion, sweet potato, taro, and the like, (d) a yeast and/or lactic acid bacterium-fermented product of flower and leaf vegetables including cabbage, perilla, mulberry leaf, fish mint, mugwort, Sasa veitchii, and dandelion, (e) a yeast and/or lactic acid bacterium-fermented product of marine algae including kombu, wakame, and mozuku, (f) a yeast and/or lactic acid bacterium-fermented product of seeds including black sesame, walnut, and ginkgo nut, and (g) a yeast and/or lactic acid bacterium-fermented product of mushrooms including maitake and shiitake, for searching for a substance which inhibits the resistance mechanism of a multidrug-resistant gram-negative bacterium and with which an existing antibacterial drug exhibits the effect thereof.

### TECHNICAL FIELD

The present invention relates to a metallo-β-lactamase inhibitor and a use thereof.

### BACKGROUND ART

Infections with multidrug-resistant gram-negative bacteria are Serious global issues because existing antibacterial drugs are hardly effective.

In particular, carbapenem antibiotics, which have been used as therapeutic agents for multidrug-resistant bacterium infections as the last resort, are decomposed by metallo-β-lactamase (NPL 1) and thus have a problem because infections with multidrug-resistant gram-negative bacteria cannot be treated.

### CITATION LIST

### Patent Literature

PTL 1: WO2016/093104

PTL 2: WO2019/009437

PTL 3: WO2019/009438

PTL 4: WO2022/003749

### Non Patent Literature

NPL 1: M Tilahun et al. Emerging Carbapenem-Resistant Enterobacteriaceae Infection, Its Epidemiology and Novel Treatment Options: A ReviewInfectin and Drug Resistance 2021 Oct 21; 14:4363-4374.

### SUMMARY OF INVENTION

### Technical Problem

Thus, a problem of the invention is to search for a substance which inhibits the resistance mechanism of a multidrug-resistant gram-negative bacterium and with which an existing antibacterial drug exhibits the effect thereof.

### Solution to Problem

As a result of extensive studies to solve the problem, the present inventors have found that the fermented plant products which the present inventors have developed so far and which have been found to have a senescence-retarding action (PTL 1), a spontaneous cancer prophylactic multidrug-resistant gram-negative and enable an existing antibacterial drug to exhibit the effect thereof, and the inventors have thus completed the invention.

That is, the invention is a metallo-β-lactamase inhibitor containing a fermented plant product which is a mixture of (a) to (g) below as an active ingredient:
(a) a koji mold-fermented product of beans and grains including barley, black soybean, red rice, black rice, adzuki bean, adlay, Japanese barnyard millet, foxtail millet, and proso millet;
(b) a yeast and/or lactic acid bacterium-fermented product of fruits including mandarin orange, grape, apple, crimson glory vine, peach, persimmon, papaya, pear, watermelon, Japanese apricot, fig, Chinese quince, pumpkin, kumquat, yuzu, loquat, apricot, jujube, chestnut, silvervine, and Japanese plum;
(c) a yeast and/or lactic acid bacterium-fermented product of root vegetables and tubers including purple sweet potato, Jerusalem artichoke, carrot, onion, sweet potato, taro, Japanese yam, daikon, red turnip, burdock, lotus root, yacon, lily bulb, arrowhead, ginger, garlic, and turmeric;
(d) a yeast and/or lactic acid bacterium-fermented product of flower and leaf vegetables including cabbage, perilla, mulberry leaf, fish mint, mugwort, Sasa veitchii, and dandelion;
(e) a yeast and/or lactic acid bacterium-fermented product of marine algae including kombu, wakame, and mozuku;
(f) a yeast and/or lactic acid bacterium-fermented product of seeds including black sesame, walnut, and ginkgo nut; and
(g) a yeast and/or lactic acid bacterium-fermented product of mushrooms including maitake and shiitake.

Moreover, the invention is an anti-multidrug-resistant gram-negative bacterium food containing the metallo-β-lactamase inhibitor.

Furthermore, the invention is an anti-multidrug-resistant gram-negative bacterium composition containing the metallo-β-lactamase inhibitor and an antibacterial drug.

In addition, the invention is a method for controlling a multidrug-resistant gram-negative bacterium characterized by administering an antibacterial drug and the metallo-β-lactamase inhibitor to a multidrug-resistant gram-negative bacterium.

Furthermore, the invention is a method for treating a multidrug-resistant gram-negative bacterium-related disease characterized by administering an antibacterial drug and the metallo-β-lactamase inhibitor to an animal having a multidrug-resistant gram-negative bacterium-related

### Advantageous Effects of Invention

The metallo-β-lactamase inhibitor of the invention contains, as an active ingredient, a specific fermented plant product which has long been used in foods so far and thus can be used safely for treatment such as control and therapy of a multidrug-resistant gram-negative bacterium or for an application such as an anti-multidrug-resistant gram-negative bacterium food.

In particular, the metallo-β-lactamase inhibitor of the invention is suitable for treating a multidrug-resistant gram-negative bacterium-related disease such as an infection.

### BRIEF DESCRIPTION OF DRAWINGS

FIG. 1 is a figure showing the measurement of (1) Minimum Inhibitory Concentration in Example 1.

FIG. 2 is a figure showing the results of (2) Combination Test in Example 1 (in the figure, the upper left shows the plate with the fermented plant product of 0, the upper right shows the plate with the fermented plant product of 1/10, the lower left shows the information on the kinds and the concentrations of the antibacterial drugs, and the lower right shows the plate with the fermented plant product (gold label) of 1/16.) FIG. 3 is a figure showing the results of (3) Checkerboard Test in Example 1.

FIG. 4 is a figure showing the results of the nitrocefin test.

### DESCRIPTION OF EMBODIMENTS

The metallo-β-lactamase inhibitor of the invention contains the fermented plant product below as an active ingredient. The fermented plant product is a mixture of the fermented plant products of (a) to (g) below.
(a) A fermented product obtained by causing a koji mold to act on beans and grains including barley, black soybean, red rice, black rice, adzuki bean, adlay, Japanese barnyard millet, foxtail millet, and proso millet.
(b) A fermented product obtained by causing a yeast and/or a lactic acid bacterium to act on fruits including mandarin orange, grape, apple, crimson glory vine, peach, persimmon, papaya, pear, watermelon, Japanese apricot, fig, Chinese quince, pumpkin, kumquat, yuzu, loquat, apricot, jujube, chestnut, silvervine, and Japanese plum, preferably a fermented
(c) A fermented product obtained by causing a yeast and/or a lactic acid bacterium to act on root vegetables and tubers including purple sweet potato, Jerusalem artichoke, carrot, onion, sweet potato, taro, Japanese yam, daikon, red turnip, burdock, lotus root, yacon, lily bulb, arrowhead, ginger, garlic, and turmeric, preferably a fermented product obtained by causing a lactic acid bacterium to act on the root vegetables and the tubers.
(d) A fermented product obtained by causing a yeast and/or a lactic acid bacterium to act on flower and leaf vegetables including cabbage, perilla, mulberry leaf, fish mint, mugwort, Sasa veitchii, and dandelion, preferably a fermented product obtained by causing a lactic acid bacterium to act on the flower and leaf vegetables.
(e) A fermented product obtained by causing a yeast and/or a lactic acid bacterium to act on marine algae including kombu, wakame, and mozuku, preferably a fermented product obtained by causing a yeast to act on the marine algae.
(f) A fermented product obtained by causing a yeast and/or a lactic acid bacterium to act on seeds including black sesame, walnut, and ginkgo nut, preferably a fermented product obtained by causing a yeast to act on the seeds.
(g) A fermented product obtained by causing a yeast and/or a lactic acid bacterium to act on mushrooms including maitake and shiitake, preferably a fermented product obtained by causing a lactic acid bacterium to act on the mushrooms.

The metallo-β-lactamase inhibitor of the invention may contain the fermented plant product obtained by mixing the fermented products of (a) to (g) above as it is as an active ingredient, but the taste and the formulation property can be improved further through multistage fermentation.

The yeast may be a yeast belonging to Saccharomyces, Zygosaccharomyces, or the like, and in particular, Saccharomyces cerevisiae (S. cervisiae), Zygosaccharomyces rouxii (Z. rouxii), Saccharomyces exiguus (S. exiguus), and the like are preferably used. Examples of the lactic acid bacterium include lactic acid bacteria belonging to Pediococcus, Leuconostoc, Lactobacillus, Lactococcus, and the like, and of these, Pediococcus acidilacti(P. acidilacti),Lactobacillus brevis(L. brevis), Leuconostoc mesenteroides(L. mesenteroides), Lactobacillus plantarum(L. plantarum), Lactococcus lactis(L. lactis), Lactobacillus sakei(L. sakei), Pediococcus (P. Lactobacillus casei(L. casei), and the like are preferably used. One species thereof or two or more species thereof can be used. Examples of the koji mold include Aspergillus oryzae, Aspergillus niger, Aspergillus kawachii, and the like, and one species thereof or two or more species thereof can be used. As the yeast, the lactic acid bacterium, and the koji mold, those commercially available, those obtained from a depository institution or the like, and the like can be used.

The plants such as the beans, the grains, and the fruits which are subjected to fermentation may be used directly or may be subjected to pretreatment, such as crushing and drying, according to the need. According to the need, the plants may be diluted by adding water.

The fermented products of (a) to (g) above are obtained by inoculating the lactic acid bacterium, the yeast, or the koji mold in the plants as raw materials and culturing. The culture may be conducted by a general method. For example, the lactic acid bacterium, the yeast, or the koji mold in an amount of about 0.001 to 1 mass % may be added to one kind of plant or a mixture of two or more kinds of plants, and fermentation treatment may be conducted at 20 to 50 °C for about 70 to 140 hours.

The fermented products of (a) to (g) obtained in this manner can be mixed and directly used as an active ingredient but are preferably further cultured at 20 to 40 °C for about 200 to 300 hours according to the need. The mixture is preferably further subjected to post-fermentation (aging). In the post-fermentation, an acetic acid bacterium may be caused to act according to the need, and for example, an acetic acid-fermented product obtained by causing the yeast to act on one kind or two or more kinds of the plants included in (a) to (g) above and then causing an acetic acid bacterium such as Acetobacter aceti(A. aceti) to act may be added. The post-fermentation may be conducted at 25 to 35 °C for 70 hours to about one year. Through an aging process by the post-fermentation, the taste and the formulation property can be improved, and the antioxidative activity can also be increased.

An example of a preferable method for producing the fermented plant product is a multistage composite fermentation method. In this method, a composite lactic acid bacterium-fermented product containing fruits, vegetables, and marine algae as main raw materials and a yeast-fermented product containing vegetables, root vegetables, seeds, mushrooms, and fruits as main raw materials are mixed. A koji mold-fermented product containing grains and beans as main raw materials is added to the mixture, and an acetic acid-fermented product is further added and mixed. Then, the mixture is concentrated by filtration and further subjected to post-fermentation for about one year. It is believed that, through such multistage composite fermentation, because the fermentation products are each further assimilated or converted by another type of strain, the increased.

The fermented plant product as the active ingredient used for the metallo-β-lactamase inhibitor of the invention has the properties of (1) to (4) below.

### (1) Taste

The fermented plant product has sweetness derived from the raw materials such as fruits and vegetables and sweetness derived from koji as well as from organic acids. Although polyphenols derived from the raw materials are contained, bitterness is weak.

### (2) Solubility in Water

The fermented plant product dissolves easily in water.

### (3) Stability

The fermented plant product is stable against heat and an acid, and a paste thereof does not spoil or change in the taste even after storage for one year at room temperature.

### (4) Safety

The vegetables, the fruits, the herbs, and the like used as the raw materials are consumed daily, and the yeasts, the lactic acid bacteria, and the koji molds used for fermentation are all strains derived from brewing, pickling, or the like of foods and thus have long been used in foods.

The fermented plant product used in the invention has the properties of (A) to (D) below.

### (A) General Ingredients (per 100 g)

Water 15 to 35 g
Protein 5 to 20 g
Lipid 1 to 8 g
Ash content 1 to 5 g
Carbohydrate 30 to 70 g
Sodium 40 to 150 mg
Vitamin B 6 0.1 to 0.5 mg
Energy 200 to 500 kcal

### (B) Amino Acid Composition (per 100 g)

Arginine 0.2 to 0.6 g
Lysine 0.1 to 0.7 g
Histidine 0.1 to 0.4 g
Phenylalanine 0.2 to 0.8 g
Tyrosine 0.1 to 0.6 g
Leucine 0.3 to 1.2 g
Isoleucine 0.2 to 0.8 g
Methionine 0.05 to 0.3 g
Valine 0.2 to 0.9 g
Serine 0.2 to 0.8 g
Threonine 0.2 to 0.7 g
Aspartic acid 0.4 to 1.5 g
Tryptophan 0.03 to 0.15 g
Cystine 0.05 to 0.40 g

### (C) Organic Acid Composition (per 100 g)

Citric acid 0.5 to 1.2 g
Malic acid 0.05 to 0.5 g
Succinic acid 0.04 to 0.3 g
Lactic acid 0.5 to 6.0 g
Formic acid 0.01 to 0.1 g
Pyruvic acid 0.005 to 0.05 g
Free γ-aminobutyric acid 0.01 to 0.05 g

### (D) Mineral Composition (per 100 g)

Phosphorus 100 to 400 mg
Iron 1 to 5 mg
Calcium 500 to 900 mg
Potassium 600 to 1000 mg
Magnesium 70 to 120 mg
Zinc 0.8 to 1.6 mg
Iodine 1.0 to 2.5 mg

Because the fermented plant product has a metallo-β-lactamase inhibitory action, a metallo-β-lactamase inhibitor can be obtained using the fermented plant product as an active ingredient. The metallo-β-lactamase that the fermented plant product can inhibit is not particularly limited and may be NDM metallo-β-lactamase, IMP metallo-β-lactamase, or the like, but NDM metallo-β-lactamase, which causes concerns of global geographic diffusion and heterologous diffusion to bacteria of Enterobacteriaceae including various pathogens, is preferable.

The fermented plant product explained above has an inhibitory action on metallo-β-lactamase and thus can be used as a metallo-β-lactamase inhibitor. Here, whether the fermented plant product has an inhibitory action on metallo-β-lactamase can be examined by the tests described in the Examples. Moreover, the inhibitory action of the fermented plant product on metallo-β-lactamase exhibits a synergistic effect with several micrograms of an antibacterial drug when the amount thereof in terms of protein is approximately 20 Mg or more, preferably 30 to 60 Mg.

The fermented plant product may be used directly for the metallo-β-lactamase inhibitor of the invention or may be formulated by adding a carrier, an excipient, a water activity modifier, and the like which are method. The fermented plant product may be concentrated to adjust the concentration or pulverized by spray-or freeze-drying or the like according to the need. As the carrier or the excipient which is used for formulation, for example, lactose, glucose, sucrose, starch, a sugar mixture, or the like is used. The final form may be a solution, paste, soft capsules, chewable, capsules, powder, granules, syrup, an oral liquid preparation, spray, ointment, cream, an external liquid preparation, eye drops, nasal drops, suppository, a plaster, or the like. Examples of the dose/usage include oral intake of about 0.1 g or more, preferably 0.1 to 12 g, more preferably 0.6 to 10 g (in terms of solid content) per day for an adult as the fermented plant product as the active ingredient. Commercially available products obtained by formulating such a fermented plant product and forming into a food include Amou Kouso Gold Label, Amou Kouso capsules, and Amou Kouso (all manufactured by Nihon Shizen Hakko Co., Ltd.), and the like.

The metallo-β-lactamase inhibitor of the invention can treat a multidrug-resistant gram-negative bacterium-related disease when the metallo-β-lactamase inhibitor is administered together with an antibacterial drug to an animal having multidrug-resistant gram-negative bacterium-related disease, preferably an animal having a multidrug-resistant gram-negative bacterium-related disease excluding human. The multidrug-resistant gram-negative bacterium-related disease is not particularly limited, but examples of life-threatening severe diseases include diseases such as pneumonia and septicemia. The antibacterial drug is not particularly limited, but examples thereof include penicillin antibiotics such as benzylpenicillin potassium (PCG), oxacillin sodium (MPIPC), ampicillin sodium (ABPC), amoxicillin (AMPC), piperacillin sodium (PIPC), and sultamicillin tosylate (SBTPC), cephem antibiotics such as cefalotin sodium (CET), cefazolin sodium (CEZ), cefotiam hydrochloride (CTM), cefoperazone sodium (CPZ), cefotaxime sodium (CTX), ceftizoxime sodium (CZX), cefmenoxime hydrochloride (CMX), ceftazidime (CAZ), ceftriaxone sodium (CTRX), cefpirome sulfate (CPR), cefepime hydrochloride (CFPM), cefozopran hydrochloride (CZOP), cefalexin (CEX), cefaclor (CCL), cefroxadine (CXD), cefixime (CFIX), ceftibuten (CETB), cefdinir (CFDN), cefuroxime sodium (CXM), cefpodoxime sodium (CPDX), cefteram hydrochloride (CFTM), cefcapene sodium (CFPN), and cefditoren sodium (CDTR), carbapenem antibiotics such as imipenem (IPM) , meropenem trihydrate (MEPM), biapenem (BIPM), doripenem hydrate (DRPM), and tebipenem hydrate (TBPM), and the like.

The method for administering the metallo-β-lactamase inhibitor of the invention to an animal having a multidrug-resistant gram-negative bacterium-related disease is not particularly limited and may be an external method or an internal method.

The metallo-β-lactamase inhibitor of the invention inhibits the resistance mechanism of a multidrug-resistant gram-negative bacterium and enables an existing antibacterial drug to exhibit the effect thereof. Accordingly, when the antibacterial drug and the metallo-β-lactamase inhibitor of the invention are administered to a multidrug-resistant gram-negative bacterium, the multidrug-resistant gram-negative bacterium can be controlled. Here, the multidrug-resistant gram-negative bacterium refers to a gram-negative bacterium which has resistance to the antibacterial drug above and whose growth is not inhibited by the antibacterial drug.

For the treatment of the multidrug-resistant gram-negative bacterium-related disease or the control of the multidrug-resistant gram-negative bacterium above, an anti-multidrug-resistant gram-negative bacterium composition containing the metallo-β-lactamase inhibitor of the invention and the antibacterial drug may also be used. The antibacterial drug content of the composition is not particularly limited and may be an amount in which the antibacterial drug generally exhibits the effect thereof.

The anti-multidrug-resistant gram-negative bacterium composition of the invention above can be formulated, like the metallo-β-lactamase inhibitor of the invention. The final form may be a solution, paste, soft capsules, chewable, capsules, powder, granules, syrup, an oral liquid preparation, spray, ointment, cream, an external liquid preparation, eye drops, nasal drops, suppository, a plaster, or the like, like the metallo-β-lactamase inhibitor of the invention.

The metallo-β-lactamase inhibitor of the invention is also a food ingredient which has long been used in foods and thus can be used as an anti-multidrug-resistant gram-negative bacterium food. For the food, the fermented plant product which is the active ingredient of the metallo-β-lactamase inhibitor of the invention is simply used as a part of raw materials or seasonings of a conventionally known food. Examples of specific foods include seasonings such as miso, breads such as batch loaf, snacks and confectionery such as rice cracker, cookies, chocolate, candy, sweetened bean paste buns, and cakes, dairy products such as yogurt and cheese, pickles such as yellow picked daikon, noodles such as ramen noodles, soba noodles, and udon noodles, soups such as corn potage and wakame soup, soft drinks, health drinks, carbonated drinks, fruit juices, and the like.

### EXAMPLES

The invention will be explained in detail below referring to Examples of the invention, but the invention is not limited to the Examples.

### Production Example 1

### Production of Fermented Plant Product:

The plants below were used as raw materials.
(a) Beans and grains (barley, black soybean, red rice, black rice, adzuki bean, adlay, Japanese barnyard millet, foxtail millet, and proso millet)
(b) Fruits (mandarin orange, grape, apple, crimson glory vine, peach, persimmon, papaya, pear, watermelon, Japanese apricot, fig, Chinese quince, pumpkin, kumquat, yuzu, loquat, apricot, jujube, chestnut, silvervine, and Japanese plum)
(c) Root vegetables (purple sweet potato, Jerusalem artichoke, carrot, onion, sweet potato, taro, Japanese yam, daikon, red turnip, burdock, lotus root, yacon, lily bulb, arrowhead, ginger, garlic, and turmeric)
(d) Flower and leaf vegetables (cabbage, perilla, mulberry leaf, fish mint, mugwort, Sasa veitchii, and dandelion)
(e) Marine algae (kombu, wakame, and mozuku)
(f) Seeds (black sesame, walnut, and ginkgo nut)
(g) Mushrooms (maitake and shiitake)

A culture solution of lactic acid bacteria (P. acidilacti, L. brevis, L. mesenteroides, L. plantarum, L. lactis, L. sakei, and L. casei) prepared at a bacterial concentration of about 1.0×10⁵ cfu/g was inoculated at 0.2 masso to the raw materials (730 g) of (c), (d), and (g) above, and the lactic acid bacteria were cultured at 30 °C for 50 hours. Moreover, a culture solution of yeasts (five strains of S. cervisiae and two strains of Z. rouxii) prepared at a bacterial concentration of about 1.0×10⁵ cfu/g was inoculated at 0.4 mass % to the raw materials (900 kg) of (b), (e), and (f) above, and the yeasts were cultured at 30 °C for 50 hours. Koji molds (yellow koji mold, black koji mold, and white koji mold) were inoculated at 0.1 masse to the (a) bean and grain raw materials (1000 kg) and cultured at 35 °C for 70 hours. Then, the cultures were mixed and then cultured at 30 °C for 200 hours. The moromi after the completion of fermentation was subjected to solid-liquid separation operation, and a paste obtained by concentrating the obtained filtrate was dispensed in containers and further subjected to post-fermentation (aging) for one year. Thus, a fermented plant product was obtained.

The fermented plant product obtained in Production Example 1 had the properties below.

### (A) General Analysis Values (per 100 g)

Water 25.2 g
Protein 11.8 g
Lipid 3.6 g
Ash content 2.1 g
Carbohydrate 57.3 g
Sodium 54.0 mg
Vitamin B 6 0.20 mg

### (B) Amino Acid Composition (per 100 g)

The sample was hydrolyzed with 6 N hydrochloric acid, and the hydrolysate was analyzed with an automated amino acid analyzer. For cystine, the sample subjected to performic acid oxidation treatment and then to hydrolysis with hydrochloric acid was used. For tryptophan, high-performance liquid chromatography was used.
Arginine 0.33 g
Lysine 0.34 g
Histidine 0.22 g
Phenylalanine 0.51 g
Tyrosine 0.32 g
Leucine 0.74 g
Isoleucine 0.42 g
Methionine 0.13 g
Valine 0.54 g
Alanine 0.48 g
Glycine 0.42 g
Proline 0.92 g
Glutamic acid 2.25 g
Serine 0.4 g
Threonine 0.36 g
Aspartic acid 0.84 g
Tryptophan 0.06 g
Cystine 0.15 g

### (C) Organic Acid Composition (per 100 g)

Citric acid 0.81 g
Malic acid 0.31 g
Succinic acid 0.12 g
Lactic acid 1.17 g
Formic acid 0.03 g
Pyruvic acid 0.01 g
Free γ-aminobutyric acid 24 mg

### (D) Mineral Composition (per 100 g)

Phosphorus 262 mg
Iron 2.65 mg
Calcium 72.1 mg
Potassium 798 mg
Magnesium 97.8 mg
Zinc 1.19 mg
Iodine 1.7 mg

The fermented plant product was the same as Amou Kouso Gold Label (manufactured by Nihon Shizen Hakko Co., Ltd.), and this was used in the Examples below.

### Example 1

### Physical Properties of Fermented Plant Product Prepared in Production Example 1:

### (1) Measurement of Minimum Inhibitory Concentration

The fermented plant product prepared in Production Example 1 was weighed, and twice the amount of sterilized water was added. The components were eluted by penetration overnight, and the supernatant after centrifugation was filtered with a filter of 0.22 μm to obtain a sample. Using the sample, the MIC against a multidrug-resistant gram-negative bacterium was examined.

As a result, the MIC of the fermented plant product against the multidrug-resistant gram-negative bacterium was ¼ concentration.

### (2) Combination Test

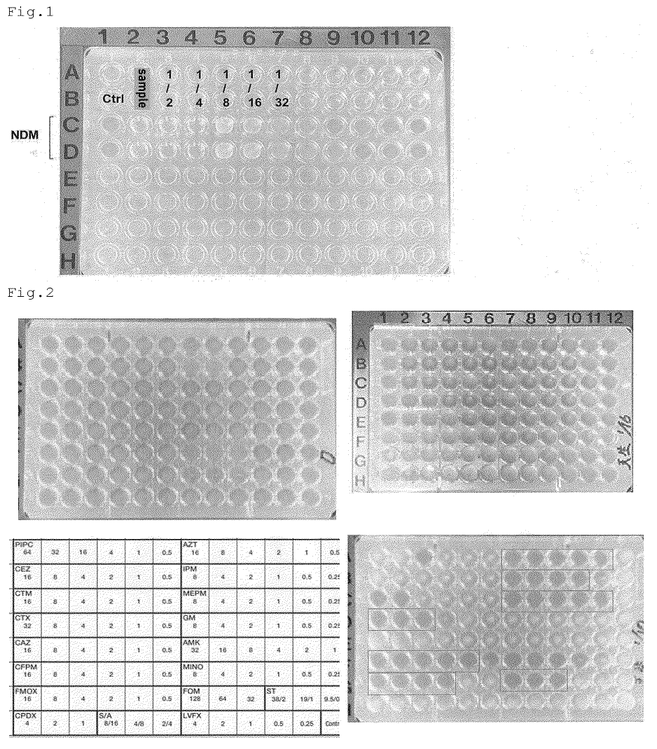

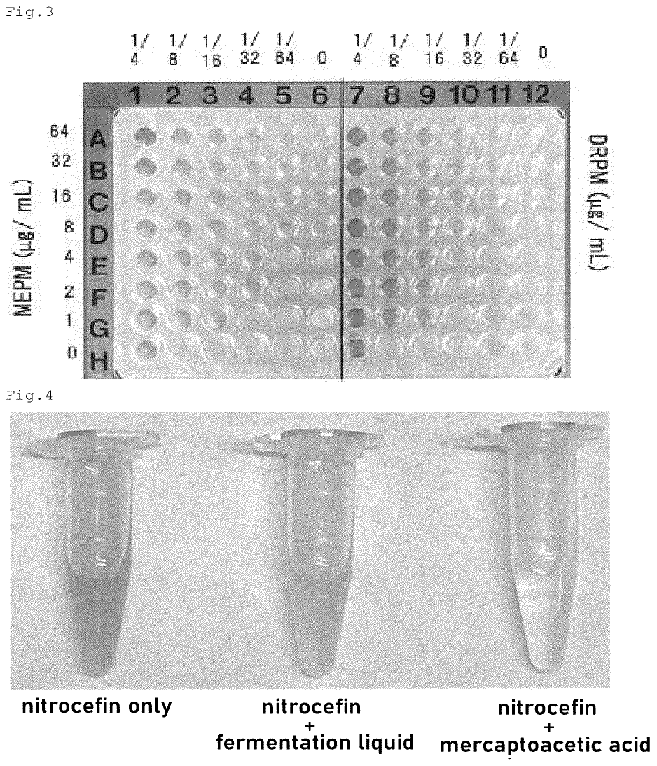

Using drug sensitivity testing plates (Dry Plate Eiken: manufactured by Eiken Chemical Co., Ltd.), in which various antibacterial drugs with a concentration gradient were immobilized, and a multidrug-resistant gram-negative bacterium, a combination test was conducted. The results are shown in FIG. 2. Here, the fermented plant product (sample) at 0, 1/10, or 1/16 was added to the plates, and a certain amount of the multidrug-resistant gram-negative bacterium was added.

As a result, although the antibacterial drugs alone did not show any effect on the multidrug-resistant gram-negative bacterium, when the fermented plant product was added at ⅛ to 1/10, which was lower than the MIC, the growth of the multidrug-resistant gram-negative bacterium was inhibited by carbapenem antibiotics such as meropenem and doripenem and also by cephem antibiotics such as cefepime, and the MIC decreased greatly.

### (3) Checkerboard Assay

A checkerboard test was conducted using meropenem (MEPM) and doripenem (DRPM) , which are relatively highly reproducible in the combination test, to examine the effect of combination use of the fermented plant product. In the checkerboard test, the antibacterial drugs and the fermented plant product were arranged with a concentration gradient on a 96-well plate, and a multidrug-resistant gram-negative bacterium was added and cultured overnight. The FIC index of each well was calculated to determine the effect of combination use. The results of the checkerboard test are shown in FIG. 3. Here, to determine the synergistic/additive effect, the FIC index (fractional inhibitory concentration) was determined by the following calculation equation, and an FIC index of 0.5 or less was defined as “with synergistic effect”.

FIC index=(MIC of combination use)/(MIC of antibacterial drug alone)+(MIC of combination use)/(MIC of fermented plant product alone) [Math. 1]

As a result, although the antibacterial drugs alone did not show any effect on the multidrug-resistant gram-negative bacterium, when the fermented plant product was added at ⅛ to 1/10, the growth of the multidrug-resistant gram-negative bacterium was inhibited by carbapenem antibiotics such as meropenem and doripenem and also by cephem antibiotics such as cefepime, and the MIC decreased greatly.

### (4) Nitrocefin Test

To examine the activity of metallo-β-lactamase, a nitrocefin test was conducted according to a general method (Yoshikazu Ishii, et. al., “Novel method to determine β-lactamase activity in sputum”, Japanese Journal of Chemotherapy, Vol. 47, No. 10, p619-622 (1999)). Because development of red color due to decomposition of nitrocefin was suppressed in the presence of the fermented plant product (the fermentation liquid in the figure) compared to the positive control, existing antibacterial drugs such as carbapenem antibiotics exhibited the efficacy due to a decrease in the activity of metallo-β-lactamase (FIG. 4) . Here, mercaptoacetic acid is a conventionally known in vitro metallo-β-lactamase inhibitor.

### Example 2

### Preparation of Anti-Multidrug-Resistant Gram-Negative Bacterium Composition:

After mixing 10 g of the fermented plant product prepared in Example 1 and 0.5 g of an antibacterial drug (meropenem) and diluting the mixture with water, the taste was adjusted, and thus a liquid anti-multidrug-resistant gram-negative bacterium composition was prepared.

### Example 3

### Preparation of Anti-Multidrug-Resistant Gram-Negative Bacterium Composition:

The fermented plant product prepared in Example 1 in an amount of 10 g and 0.5 g of an antibacterial drug (meropenem) were mixed, and thus a paste-like anti-multidrug-resistant gram-negative bacterium composition was prepared.

### Example 4

### Anti-Multidrug-Resistant Gram-Negative Bacterium Food (Miso-Like Food):

The fermented plant product produced in Production Example 1 and miso were mixed, and the taste was adjusted with a seasoning. Thus, an anti-multidrug-resistant gram-negative bacterium food (miso-like food) was produced.

### Example 5

### Anti-Multidrug-Resistant Gram-Negative Bacterium Food (Drink):

After dissolving the fermented plant product produced in Production Example 1 in water, a high fructose corn syrup (fructose content of 50%

### Example 6

### Anti-Multidrug-Resistant Gram-Negative Bacterium Food (Soft Capsules):

The fermented plant product produced in Production Example 1, vegetable oil, and lecithin were mixed, and soft capsules were filled with the mixture by a general method. Thus, an anti-multidrug-resistant gram-negative bacterium food (soft capsules) was produced.

### Example 7

### Anti-Multidrug-Resistant Gram-Negative Bacterium Food (Chewable Tablets):

The fermented plant product produced in Production Example 1, crystalline cellulose, and white sugar were mixed, and the mixture was tableted by a general method. Thus, an anti-multidrug-resistant gram-negative bacterium food (chewable tablets) used for improving QOL was produced.

### Example 8

### Anti-Multidrug-Resistant Gram-Negative Bacterium Food (Soft Capsules):

The fermented plant product produced in Production Example 1 (daily dose), vegetable oil, lecithin, and 900 mg of anserine were mixed, and soft capsules were filled with the mixture by a general method. Thus, an anti-multidrug-resistant gram-negative bacterium food (soft capsules) was produced.

### Industrial Applicability

The metallo-β-lactamase inhibitor of the invention inhibits the resistance mechanism of a multidrug-resistant gram-negative bacterium and enables an antibacterial drug to exhibit the effect thereof, and thus the metallo-β-lactamase inhibitor can be used for treating a disease related to a multidrug-resistant gram-negative bacterium.

1. A metallo-β-lactamase inhibitor, comprising a fermented plant product which is a mixture of (a) to (g) as an active ingredient:
(a) a koji mold-fermented product of beans and grains including barley, black soybean, red rice, black rice, adzuki bean, adlay, Japanese barnyard millet, foxtail millet, and proso millet;
(b) a yeast and/or lactic acid bacterium-fermented product of fruits including mandarin orange, grape, apple, crimson glory vine, peach, persimmon, papaya, pear, watermelon, Japanese apricot, fig, Chinese quince, pumpkin, kumquat, yuzu, loquat, apricot, jujube, chestnut, silvervine, and Japanese plum;
(c) a yeast and/or lactic acid bacterium-fermented product of root vegetables and tubers including purple sweet potato, Jerusalem artichoke, carrot, onion, sweet potato, taro, Japanese yam, daikon, red turnip, burdock, lotus root, yacon, lily bulb, arrowhead, ginger, garlic, and turmeric;
(d) a yeast and/or lactic acid bacterium-fermented product of flower and leaf vegetables including cabbage, perilla, mulberry leaf, fish mint, mugwort, Sasa veitchii, and dandelion;
(e) a yeast and/or lactic acid bacterium-fermented product of marine algae including kombu, wakame, and mozuku;
(f) a yeast and/or lactic acid bacterium-fermented product of seeds including black sesame, walnut, and ginkgo nut; and
g) a yeast and/or lactic acid bacterium-fermented product of mushrooms including maitake and shiitake.

2. The metallo-β-lactamase inhibitor according to claim 1, wherein the fermented plant product comprises amino acids in the following composition per 100 g of the fermented plant product:
arginine 0.2 to 0.6 g,
lysine 0.1 to 0.7 g,
histidine 0.1 to 0.4 g,
phenylalanine 0.2 to 0.8 g,
tyrosine 0.1 to 0.6 g,
leucine 0.3 to 1.2 g,
isoleucine 0.2 to 0.8 g,
methionine 0.05 to 0.30 g,
valine 0.2 to 0.9 g,
alanine 0.2 to 0.9 g,
glycine 0.2 to 0.7 g,
proline 0.4 to 1.2 g,
glutamic acid 1.2 to 3.0 g,
serine 0.2 to 0.8 g,
threonine 0.2 to 0.7 g,
aspartic acid 0.4 to 1.5 g,
tryptophan 0.03 to 0.15 g, and
cystine 0.05 to 0.40 g.

3. The metallo-β-lactamase inhibitor according to claim 1, wherein the fermented plant product comprises organic acids in the following composition per 100 g of the fermented plant product:
citric acid 0.5 to 1.2 g,
malic acid 0.05 to 0.5 g,
succinic acid 0.04 to 0.3 g,
lactic acid 0.5 to 6.0 g,
formic acid 0.01 to 0.1 g,
pyruvic acid 0.005 to 0.05 g, and
free γ-aminobutyric acid 0.01 to 0.05 g.

4. The metallo-β-lactamase inhibitor according to claim 1, wherein the lactic acid bacterium is at least one species selected from the group consisting of Pediococcus acidilacti (P. acidilacti), Lactobacillus brevis(L. brevis), Leuconostoc mesenteroides(L. mesenteroides), Lactobacillus plantarum(L. plantarum), Lactococcus lactis(L. lactis), Lactobacillus sakei(L. sakei), Pediococcus pentosaceus(P. pentosaceus), Lactobacillus casei(L. casei), and Lactobacillus curvatus(L. curvatus).

5. The metallo-β-lactamase inhibitor according to claim 1, wherein the yeast is Saccharomyces cerevisiae (S. cervisiae) and/or Zygosaccharomyces rouxii (Z. rouxii)

6. An anti-multidrug-resistant gram-negative bacterium food comprising the metallo-β-lactamase inhibitor of claim 1.

7. An anti-multidrug-resistant gram-negative bacterium composition comprising the metallo-β-lactamase inhibitor of claim and an antibacterial drug.

8. A method for controlling a multidrug-resistant gram-negative bacterium, the method comprising:
administering an antibacterial drug and the metallo-β-lactamase inhibitor of claim to a multidrug-resistant gram-negative bacterium.

9. A method for treating a multidrug-resistant gram-negative bacterium-related disease, the method comprising:
administering an antibacterial drug and the metallo-β-lactamase inhibitor of claim 1 to an animal having a multidrug-resistant gram-negative bacterium-related disease.

10. The anti-multidrug-resistant gram-negative bacterium food of claim 6, further comprising at least one selected from the group consisting of miso, seasoning, vegetable oil, lecithin, crystalline cellulose, white sugar, and anserine.

11. The anti-multidrug-resistant gram-negative bacterium composition of claim 7, wherein the antibacterial drug is at least one selected from the group consisting of benzylpenicillin potassium (PCG), oxacillin sodium (MPIPC), ampicillin sodium (ABPC), amoxicillin (AMPC), piperacillin sodium (PIPC), and sultamicillin tosylate (SBTPC), cephem antibiotics such as cefalotin sodium (CET), cefazolin sodium (CEZ), cefotiam hydrochloride (CTM), cefoperazone sodium (CPZ), cefotaxime sodium (CTX), ceftizoxime sodium (CZX), cefmenoxime hydrochloride (CMX), ceftazidime (CAZ), ceftriaxone sodium (CTRX), cefpirome sulfate (CPR), cefepime hydrochloride (CFPM), cefozopran hydrochloride (CZOP), cefalexin (CEX), cefaclor (CCL), cefroxadine (CXD), cefixime (CFIX), ceftibuten (CETB), cefdinir (CFDN), cefuroxime sodium (CXM), cefpodoxime sodium (CPDX), cefteram hydrochloride (CFTM), cefcapene sodium (CFPN), and cefditoren sodium (CDTR), imipenem (IPM), meropenem trihydrate (MEPM), biapenem (BIPM), doripenem hydrate (DRPM), and tebipenem hydrate (TBPM).

12. The anti-multidrug-resistant gram-negative bacterium composition of claim 7, wherein the antibacterial drug is meropenem.

13. The anti-multidrug-resistant gram-negative bacterium composition of claim 7, wherein a ratio of the antibacterial drug to the metallo-β-lactamase inhibitor is from 1:10 to 1:30.

14. The method for treating a multidrug-resistant gram-negative bacterium-related disease of claim 9, further comprising:
administering a daily dose of from 0.1 to 12 g of the metallo-β-lactamase inhibitor.

15. The method for treating a multidrug-resistant gram-negative bacterium-related disease of claim 9, further comprising:
mixing the metallo-β-lactamase inhibitor with a carrier or excipient selected from the group consisting of lactose, glucose, sucrose, starch, and a sugar mixture.
